(19) United States
(12) Patent Application Publication
(10) Pub. No.: US 2021/0180003 A1
(43) Pub. Date: Jun. 17, 2021

(54) Systems and Methods for Auto-Inoculation in Seed Train and Production Processes

(71) Applicant: Regeneron Pharmaceuticals, Inc., Tarrytown, NY (US)

(72) Inventors: Mark CZETERKO, Ridgefield, CT (US); Alessandra STARLING, Mohegan Lake, NY (US); Colin ORR, New York, NY (US); William Seth PIERCE, Apex, NC (US); Matthew CONWAY, New York (GA)

(21) Appl. No.: 17/078,137

(22) Filed: Oct. 23, 2020

Related U.S. Application Data
(60) Provisional application No. 62/925,940, filed on Oct. 25, 2019.

(51) Int. Cl. C12M 1/36; C12M 1/00

(52) U.S. Cl. CPC C12M 41/48; C12M 29/00

(57) ABSTRACT

A system and method for auto-inoculating a bioreactor in a seed train process includes an expansion chamber for expanding an initial cell stock to a viable cell density, a bioreactor for inoculation with the expanded cell stock; a fluid communication path between the expansion chamber and the bioreactor; a pump for controlling fluid flow through the fluid communication path; a Raman spectrometer for generating Raman spectral data; a multivariate model providing predictions of processing variables in the expansion chamber; and a computer system for controlling the pump to effect auto-inoculation of the bioreactor from the expansion chamber, through the fluid communication path, when the computer system determines from the Raman spectral data that one or more predefined trigger events have occurred.

### CROSS REFERENCE TO RELATED APPLICATIONS

This application claims benefit of and priority to U.S. Provisional Patent Application No. 62/925,940 filed on Oct. 25, 2019, and is incorporated by reference in its entirety.

### FIELD OF THE INVENTION

Inventions encompassed hereby are inclusive of bioreactor systems, and methods for monitoring and controlling a seed train process in a bioreactor system. Particular embodiments are further inclusive of bioreactor systems that include a Raman spectrometer, and methods that use Raman spectroscopy for monitoring and controlling a seed train process.

### BACKGROUND OF THE INVENTION

Therapeutic antibodies, and in particular monoclonal antibodies (mAb), have become an important tool in modern medicine for the development of target proteins that may be used in the treatment of a wide array of illnesses, including cancer and autoimmune diseases.

Target proteins of interest are produced by a cell line that is expanded from an initial cryopreserved cell stock through a seed train process through one or more stages until a predetermined viable cell density (VCD) is achieved, at which time the expanded cell stock is then introduced to a production bioreactor for inoculating a culture medium held therein. Following inoculation, the cell culture continues to grow in the bioreactor until a target protein is expressed in a desired quantity, after which the cell culture fluid can be harvested and the target protein may be isolated and purified.

Traditional seed train processes included multiple stages of cell growth and expansion, using vessels of increasing size, between the initial cryopreserved cell stock and the final production bioreactor. In early processes the initial cryopreserved cell stock could be expanded through several stages that may include, for example, one or more shake flasks, one or more spinners, one or more wave bags, and one or more expansion chambers before the predetermined VCD is reached for inoculation of a production bioreactor. More recently, more efficient seed train processes for achieving a predetermined VCD in fewer steps have been developed. However, modern processes continue to require cell expansion through at least one expansion chamber for reaching a predetermined VCD before inoculation of a culture medium in final production bioreactor.

Generally, the final target protein concentration can be increased and batch-to-batch consistency can be reduced in a production bioreactor by using an inoculum having the same VCD. However, there is a range to the inoculum VCD that leads to a target production bioreactor performance. For example, too low an inoculum VCD can cause undesirable lactate cell metabolism, and too high an inoculum VCD can produce lower cell growth in the production bioreactor due to cells exiting exponential growth phase.

Undesirable lactate cell metabolism and lower cell growth in the production bioreactor result in lower target protein quantities produced than could have been produced from those cells, and therefore equate to a loss of throughput and an overall decrease in process efficiencies. As such, it is desirable that cell expansion be grown to a predetermined VCD that leads to desirable lactate cell metabolism in the production bioreactor and maintains exponential cell growth, and that the production bioreactor be inoculated as soon as possible after reaching the predetermined VCD.

It will be appreciated that a target VCD range will vary from one cell line to another, based on properties of the different cell lines. However, a further complication arises in that cell expansion may also vary between individual production runs of a common cell line due to variations in the culture medium and other operating conditions. As a result, the timing for inoculating a production bioreactor, following cell expansion in an upstream expansion chamber to a predetermined VCD, can be variable.

Despite the many advances provided to date in the art, there remains a need for further improvements to seed train processes for yet further advancing the state of the art, and improving throughput generally. As one non-limiting example, the state of the art would benefit from improvements that facilitate inoculation of a production bioreactor following cell expansion to a predetermined VCD.

### SUMMARY OF THE INVENTION

The present invention concerns systems and methods that use Process Analytical Technology (PAT) tools and a PAT Knowledge Manager to provide monitoring and control strategies to increase process consistency. In one aspect, systems and methods according to the present invention lessen the dependency on manual operations for obtaining and verifying offline samples to confirm target cell densities, and for initiating the transfer of a cell culture between bioreactors, such as when inoculating a final production bioreactor. Use is made of Raman spectroscopy, in conjunction with PAT data management software, to enable the continuous monitoring of cell growth and automated transfer of a cell culture between two vessels when a predefined trigger event is detected (e.g., when a target viable cell density is detected).

Systems herein operate to monitor a cell culture in an expansion chamber using a Raman spectrometer, and controlling inoculation of a production bioreactor with an inoculum from the expansion chamber based on Raman spectral data. In some examples, the system control scheme includes automatically inoculating a production bioreactor through use of an in-line pump based on a determination that a cell culture in an upstream expansion chamber (e.g., an upstream bioreactor of relatively lesser volume) has reached a predetermined viable cell density (VCD). Such systems and methods may be used with cell cultures that include mammalian cells, for example, Chinese Hamster Ovary (CHO) cells, and the cell culture may be cultivated to produce proteins that include antibodies, antigen-binding fragments thereof, or fusion proteins.

Systems here may also include one or more processors in communication with a computer readable medium (e.g., a physical, non-transitory memory) that stores software code for execution by the one or more processors for causing the system to receive data including a VCD of the cell culture from a Raman spectrometer; and for executing an inoculation of a production bioreactor based on Raman spectral data. The software code stored on the computer readable medium may be further configured to use one or more multivariate models, such as a Partial Least Squares regression model, to interpret Raman spectral data. The software code may be further configured to control the system to perform one or more signal processing techniques on the spectral data, for example, a noise reduction technique.

Systems disclosed herein operate to monitor and control a seed train process, and may include an expansion chamber for receiving an initial cell stock for expansion into a viable cell culture; a bioreactor in fluid communication with the expansion chamber for receiving a viable cell culture; a pump for effecting transfer of a viable cell culture from the expansion chamber to the bioreactor through a fluid communication path between the expansion chamber and the bioreactor; a multivariate model for correlating the Raman spectral data to one or more process variables of the cell expansion process within the expansion chamber using Raman spectrometry, the Raman spectrometer being adapted to generate Raman spectral data; and a computer system in signal communication with the Raman spectrometer for receiving Raman spectral data, and in signal communication with the pump for controlling operation of the pump for effecting transfer of a viable cell culture from the expansion chamber to the bioreactor.

The Raman spectrometer may be adapted to generate Raman spectral data and a multivariate model correlates Raman spectral data to one or more process variables, and the computer system may be adapted to compare the process variable measurements to one or more predefined process set points to determine if one or more process variable measurements have satisfied a predefined trigger value. When the computer system determines that a process variable measurement in the Raman spectral data has satisfied a predefined trigger value, the control system instructs the pump to execute an auto-transfer of a cell culture volume from the expansion chamber to the bioreactor, thereby auto-inoculating a culture medium in the bioreactor with a cell culture from the expansion chamber.

The computer system processes Raman spectral data from the Raman spectrometer to generate a multivariate model of the one or more process variables, which may include a partial least squares regression model. When comparing process variable measurements from the Raman spectral data to one or more predefined process set points, the computer system may use process variable measurements from a plurality of predefined isolated regions of the Raman spectral data, such as wavelength regions of 800-850 cm⁻¹; 1260-1470 cm⁻¹; 1650-1840 cm⁻¹; and/or 2825-3080 cm⁻¹.

Systems herein may be used to auto-inoculate a bioreactor by expanding a cell stock in the expansion chamber; generating Raman spectral data, using the multivariate model to predict one or more process variables of the cell expansion in the expansion chamber; comparing, with the computer system, process variable predictions from the Raman spectral data and predefined process set points; and actuating the pump to auto-inoculate the bioreactor with a viable cell culture from the expansion chamber when the computer system determines that one or more process variable predictions from the Raman spectral data satisfies a predefined trigger value.

Systems herein may process Raman spectral data received from the Raman spectrometer to generate a multivariate model of the one or more process variables, and may then obtain process variable predictions from the multivariate model for comparison with stored predefined trigger values. After completing a seed train process, the system may store the multivariate model for that completed seed train process for use in monitoring and controlling a subsequent seed train process. In a subsequent seed train process, the system may use one or more multivariate models from one or more prior seed train processes for comparison against one or more processing variable measurements in the subsequent seed train process. The system may use one or more multivariate models from one or more prior seed train processes for monitoring processing conditions in the expansion chamber and/or the bioreactor.

Both the foregoing general description and the following detailed description are exemplary and explanatory only and are intended to provide further explanation of the invention as claimed. The accompanying drawings are included to provide a further understanding of the invention; are incorporated in and constitute part of this specification; illustrate embodiments of the invention; and, together with the description, serve to explain the principles of the invention.

### BRIEF DESCRIPTION OF THE DRAWINGS

Further features and advantages of the invention can be ascertained from the following detailed description that is provided in connection with the drawings described below:

FIG. 1 shows one example of a system according to the present invention;

FIG. 2 shows one example of a computer architecture that may be used with the computer system of the system in FIG. 1;

FIG. 3. shows one example of a method of using the system of FIG. 1 to auto-inoculating a bioreactor;

FIG. 4 shows steps for collecting and processing spectral data, and generating a regression model from the collected spectral data, using the system of FIG. 1;

FIGS. 4a-4d. show for collecting and processing spectral data, and generating a regression model from the collected spectral data, using the system of FIG. 1;

FIG. 5. shows an example of a regression model generated from Raman spectral data using the system of FIG. 1;

FIG. 6 shows data ranges of a spectral model for use in generating a regression model using the system of FIG. 1;

FIG. 7 shows one comparative example of two regression models generated using different regions of Raman spectral data with the system of FIG. 1; and

FIG. 8 shows a weighted regression model of predicted process values generated by the system of FIG. 1.

### DETAILED DESCRIPTION OF THE INVENTION

The following disclosure discusses the present invention with reference to the examples shown in the accompanying drawings, though does not limit the invention to those examples.

As used herein, the singular forms “a,” “an,” and “the” include plural referents unless the context clearly dictates otherwise. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential or otherwise critical to the practice of the invention. Unless made clear in context, terms such as “first”, “second”, “third”, etc. when used to describe multiple devices or elements, are so used only to convey the relative actions, positioning and/or functions of the separate devices, and do not necessitate either a specific order for such devices or elements, or any specific quantity of such devices or elements.

The word “substantially”, as used herein with respect to any property or circumstance, refers to a degree of deviation that is sufficiently small so as to not appreciably detract from the identified property or circumstance. The exact degree of deviation allowable in a given circumstance will depend on the specific context, as would be understood by one having ordinary skill in the art.

Use of the terms “about” or “approximately” are intended to describe values above and/or below a stated value or range, as would be understood by one having ordinary skill in the art in the respective context. In some instances, this may encompass values in a range of approx. +/−10%; in other instances there may be encompassed values in a range of approx. +/−5%; in yet other instances values in a range of approx. +/−2% may be encompassed; and in yet further instances, this may encompass values in a range of approx. +/−1%. The applicable range for each instance will be made clear by context, and no further limitation is implied.

It will be understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof, unless indicated herein or otherwise clearly contradicted by context.

Recitations of value ranges herein, unless otherwise indicated herein, serve as a shorthand for referring individually to each separate value falling within the stated ranges, including the endpoints of each range, each separate value within each range, and all intermediate ranges subsumed by each range, with each incorporated into the specification as if it were individually recited herein.

All methods described herein can be performed with the individual steps executed in any suitable order. Unless indicated herein, or otherwise clearly contradicted by context, methods may be performed in the precise order disclosed and without any intermediate steps, with one or more further steps interposed between the disclosed steps, with the disclosed steps performed in an order other than the exact order disclosed, with one or more steps performed simultaneously, and with one or more disclosed steps omitted.

The terms, “cell culture” and “cell culture media” may be used interchangeably and include any solid, liquid, or semi-solid designed to support the growth and maintenance of microorganisms, cells, or cell lines. Components such as polypeptides, sugars, salts, nucleic acids, cellular debris, acids, bases, pH buffers, oxygen, nitrogen, agents for modulating viscosity, amino acids, growth factors, cytokines, vitamins, cofactors, and nutrients may be present within the cell culture media. Some examples may provide a mammalian cell culture process using mammalian cells or cell lines, such as a Chinese Hamster Ovary (CHO) cell line grown in a chemically defined basal medium.

As used herein, the term “nutrient” may refer to any compound or substance that provides nourishment essential for growth and survival of a cell culture. Examples of nutrients include, but are not limited to, simple sugars such as glucose, galactose, lactose, fructose, or maltose; amino acids; and vitamins, such as vitamin A, B vitamins, and vitamin E.

As used herein, the term “signal communication” may refer to any manner of communicating a signal between two or more devices including, though not limited to, physical connections (e.g., hardwire signal paths) and non-physical connections (e.g., wireless signal paths). Unless otherwise stated, signal communication between two devices may be direct (e.g., a transmitter in a first device communicating directly with a receiver in a second device) or indirect (e.g., a transmitter in a first device and a receiver in a second device communicating with one another through an intermediate transceiver).

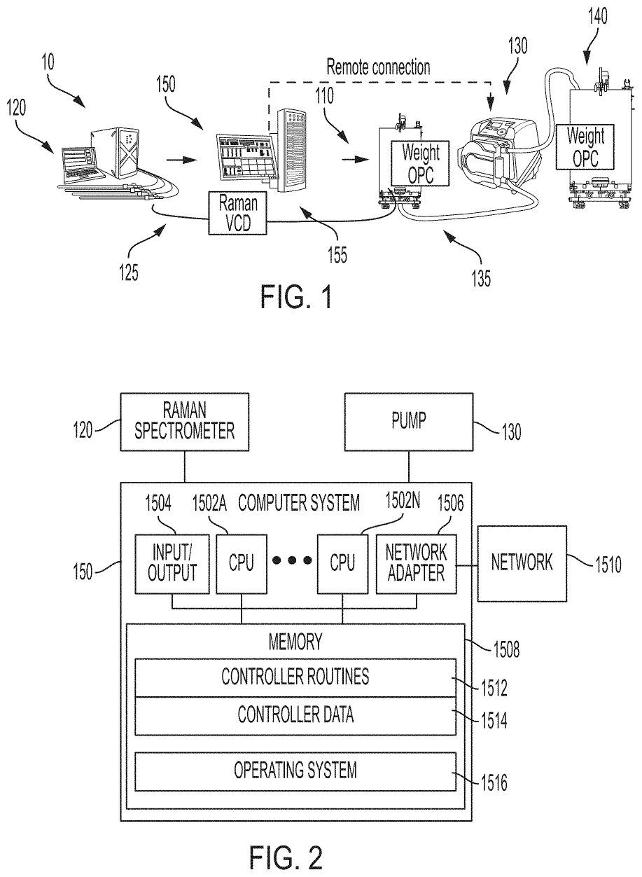

In one example, as shown in FIG. 1, there is provided a system 10 comprising an expansion chamber 110, a spectrometer 120, a pump 130, a production bioreactor 140, and a computer system 150. The expansion chamber 110 and bioreactor 140 are in fluid communication with one another via a feed line 135, with fluid flow through the feed line 135 being controlled by the pump 130. The spectrometer 120 has at least one probe 125 adapted for monitoring a cell culture within the expansion chamber 110, and is in signal communication with the computer system 150. The computer system 150 is in signal communication with at least the spectrometer 120 and the pump 130, though may also be in signal communication with one or more, or each, of the expansion chamber 110, the bioreactor 140, and a network. In some examples, two or more of the Raman spectrometer 120, the computer system 140, and the pump 130 may be provided as a single integral apparatus.

The expansion chamber 110 and bioreactor 140 may be operable as batch, fed-batch, and/or continuous units. The expansion chamber 110 and bioreactor 140 may both range in volume from about 2 L to about 10,000. As one example, the expansion chamber 110 may be a 50 L stainless steel unit and the bioreactor 140 may be a 250 L unit. Both the expansion chamber 110 and bioreactor 140 should maintain a cell count in the range of about 0.25×10⁶ cells/mL to about 100×10⁶ cells/mL.

In one example, the spectrometer 120 is a Raman spectrometer that may monitor and collect data as to any component of a cell culture that has a detectable Raman spectrum. The systems and methods herein may be used to monitor any component of a cell culture media including components added to the cell culture, substances secreted from the cells, and cellular components present upon cell death. Components of the cell culture media that may be monitored by the systems and methods include, but are not limited to: nutrients, such as amino acids and vitamins; lactate; co-factors; growth factors; cell growth rate; pH; oxygen; nitrogen; viable cell count; acids; bases; cytokines; antibodies; and metabolites.

The computer system 150 may be implemented using one or more specially programmed general-purpose computer systems, such as embedded processors, systems on a chip, personal computers, workstations, server systems, and minicomputers or mainframe computers, or in distributed, networked computing environments. The computer system 150 may include one or more processors (CPUs) 1502A,-1502N, input/output circuitry 1504, network adapter 1506, and memory 1508. CPUs 1502A-1502N execute program instructions in order to carry out the functions of the present systems and methods. Typically, CPUs 1502A-1502N are one or more microprocessors, such as an INTEL CORE® processor.

Input/output circuitry 1504 provides the capability to input data to, or output data from, the computer system 150. For example, input/output circuitry 1504 may include input devices, such as keyboards, mice, touchpads, trackballs, scanners, analog to digital converters, etc., output devices, such as video adapters, monitors, printers, etc., and input/output devices, such as, modems, etc. Network adapter 1506 interfaces the computer system 150 with a network 1510, which may be any public or proprietary LAN or WAN, including, but not limited to the Internet.

Memory 1508 stores program instructions that are executed by, and data that are used and processed by, CPUs 1502A-1502N to perform the functions of computer system 150. Memory 1508 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a fiber channel-arbitrated loop (FC-AL) interface.

Memory 1508 may include controller routines 1512, controller data 1514, and operating system 1516. Controller routines may include software to perform processing to implement one or more controllers. Controller data may include data needed by controller routines to perform processing. In one embodiment, controller routines may include multivariate software for performing multivariate analysis, such as PLS regression modeling. In this aspect, controller routines may include SIMCA (Sartorius Stedim Data Analytics AB, Umea, Sweden) for performing PLS modeling. In another embodiment, controller routines may also include software for performing noise reduction on a data set. In this aspect, the controller routines may include MATLAB Runtime (The Mathworks Inc., Natick, Mass.) for performing noise reduction filter models. Moreover, controller routines may include software, such as MATLAB Runtime, for operating an automated control unit, for example, a proportional-integral-derivative (PID) controller. The software for operating the system should also be able to calculate a difference between a predefined set point and a measured process variable (for example, a measured nutrient concentration) and provide a prediction for when the predefined set point will be reached. When including functionality for predicting the time to reach a predefined set point, as with a PID controller, the computer system 150 is also in signal communication with pump 130 so that a correct amount of inoculum may be pumped into the expansion chamber 110 and/or bioreactor 140, as predefined. The system 10 may monitor and control process variables in the expansion chamber 110 and the bioreactor 140, as shown in FIG. 1, or in a plurality of expansion chambers and/or a plurality of bioreactors.

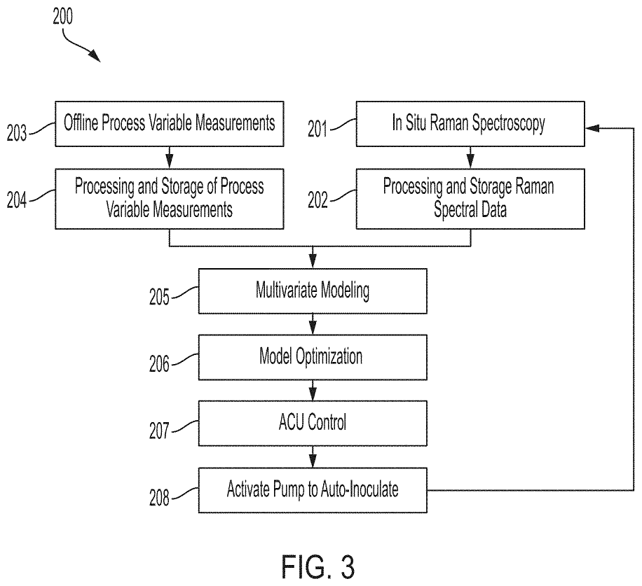

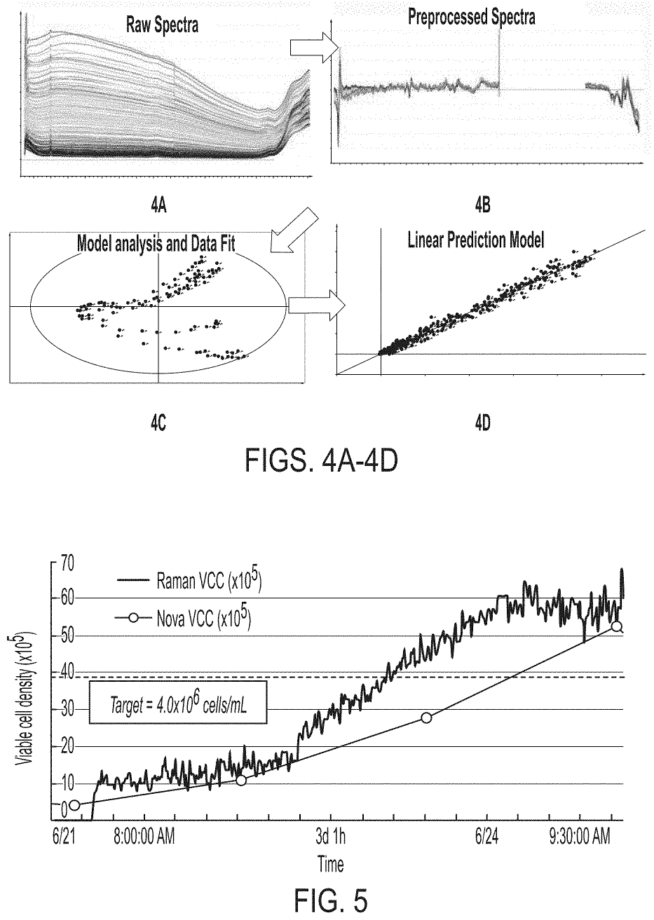

FIG. 3 shows a flow chart for one method 200 of performing a seed train process with the system 10. Following introduction of a cryopreserved cell stock into a culture medium in the expansion chamber 110, the Raman spectrometer 120 collects Raman spectral data (FIG. 4a) from the expanding cell culture in the expansion chamber 110 (step 201). Raman spectroscopy is a form of vibrational spectroscopy that provides information about molecular vibrations that can be used for sample identification and quantitation. The Raman spectrometer 120 collects Raman spectral data through a probe 125, which may be either a contact or non-contact probe. A non-contact probe 125 enables in situ Raman analysis of the cell culture without requiring contact with, or extraction from, the cell culture. In situ Raman analysis is advantageous in that it is non-invasive, and therefore reduces the risk of contaminating the cell culture, which can introduce undesirable influences on the cell culture and the resulting proteins.

Raman spectral data is acquired at a regular frequency, such that the spectral data is continuously up to date. Spectral data may be collected once about every 10 to 120 minutes, once about every 15 to 60 minutes, or once about every 20 to 30 minutes. The appropriate sampling frequency may be determined on a case-by-case basis, for example based on the particular cell line and/or processing conditions, as deemed appropriate for ensuring that the spectral data is adequately indicative of a current state of a given cell culture. Use may be made of any commercially available Raman spectrometer, non-limiting example of which may include RamanRXN2 and RamanRXN4 spectrometers (Kaiser Optical Systems, Inc. Ann Arbor, Mich.).

Following collection, in a step 202, raw spectral data is transmitted to the computer system 150, where it is preprocessed and the processed Raman data (FIG. 4b) is stored in the memory 1508 at a dedicated location for subsequent use (step 202). In some examples, processing of the Raman spectral data includes the application of one or more spectral filters to correct any baseline shifts. For example, raw spectral data may be treated with a point smoothing technique or a normalization technique. Normalization may be needed to correct for any laser power variation and exposure time by the Raman spectrometer. In some examples the raw Raman spectral data may be treated with point smoothing, such as 1^{st} derivative with 21 cm⁻¹ point smoothing, and normalization, such as Standard Normal Variate (SNV) normalization.

In parallel with the collection of Raman spectral data (step 201), process variable data is also collected through an alternative, “offline” method (step 203) and likewise stored in the memory 1508 (step 204). The offline process variable data may be collected through any appropriate analytical method, for example, through a manually obtained sample of the cell culture that is tested in a local analyzer, such as a BioProfile Flex® Analyzer (Nova Biomedical Corporation, Massachusetts, USA). Offline process variable data is collected at a lower frequency than the Raman spectral data—e.g., once about every 24 hours, once about every 12 hours, or once about every 6 hours—and serves as a baseline reference for the Raman spectral data. When collected, offline process variable data is also stored in the computer system 150 at a dedicated location. When collecting offline process variable data, the computer system 150 may also store information from PAT and/or Data Management systems (e.g., lab data management system and/or continuous, online process data).

The computer system 150 uses the processed Raman data to generate a multivariate model that informs one or more processing variables of the cell culture (step 205). When offline process variable data is available, the computer system 150 compares the Raman spectral data with corresponding offline process variable data in order to correlate peaks between the two data sets. The computer system uses the stored PAT and/or Data Management data to correlate the offline process variable data with the corresponding Raman spectral data. Any type of multivariate software package, for example, SIMCA 13 (Sartorius Stedim Data Analytics AB, Umea, Sweden), may be used to correlate peaks between the two sets of spectral data.

The multivariate modeling performed by the computer system 150 may include, though is not limited to, Partial Least Squares (PLS), Principal Component Analysis (PCA), Orthogonal Partial least squares (OPLS), Multivariate Regression, Canonical Correlation, Factor Analysis, Cluster Analysis, Graphical Procedures, and the like. In the example shown in FIGS. 4c-4d, a PLS regression model is created by fitting available measurement values obtained from the Raman spectral data and the offline process variable data (FIG. 4c), and the model is optimized (step 206) by removing outliers to yield a linear prediction model (FIG. 4d). Such a PLS regression model may be used to provide predicted process values, for example, predicted concentration values for a particular variable to be monitored by the computer system 150 for effecting control over the system 10.

Model optimization may include the application of additional signal processing techniques to the multivariate model and the predicted process values therein. In one example, a noise reduction technique may be applied to the predicted process values to perform data smoothing and/or signal rejection. Such noise reduction techniques provide a filtered model. One noise reduction technique is to combine raw measurements with a model-based estimate of what the measurements should yield according to the model. The noise reduction technique may combine a current predicted process value with its uncertainties, which can be determined by the repeatability of the predicted process values and the current process conditions. Once the next predicted process value is observed, the estimate of the predicted process value is updated using a weighted average where more weight is given to the estimates with higher certainty. Using an iterative approach, the final process values may be updated based on the previous measurement and the current process conditions. In this aspect, the algorithm should be recursive and able to run in real time so as to utilize the current predicted process value, the previous value, and experimentally determined constants. The noise reduction technique improves the robustness of the measurements from the Raman analysis and the PLS predictions.

The computer system 150 includes an automated control unit (ACU) 155 that operates, in a step 207, to assess the modeled spectral data to determine whether the pump 130 should be activated to transfer a cell culture volume from the expansion chamber 110 to the bioreactor 140, so as to inoculate a culture media in the bioreactor 140. The ACU 155 stores one or more predefined set point values that each define a trigger event for executing an auto-inoculation. The ACU 155 may be any type of automated controller that is able to compare filtered process values with one or more predefined set point values, and to automatically execute a predefined action upon determining that one or more filtered process values satisfies a condition of a corresponding set point value (e.g., is at or above a maximum set point value; at or below a minimum set point value; or the like). If the ACU 155 determines that predefined conditions for inoculation have been met, then the ACU 155 actuates the pump 130 to effect a fluid flow through fluid line 135, thereby auto-inoculating the bioreactor 140 (step 208), otherwise the process returns to the data collection through an iterative loop (step 209).

In one example, the ACU 155 stores a predefined set point value (also referred to herein as a “trigger value”) based on a target VCD for a cell culture that is the subject of a current seed train process. Such a predefined set point value may be set to a target VCD, such that there may be desirable cell metabolism in the production bioreactor while also maintaining the cells in exponential growth phase. For example, a VCD-based trigger value may be set to a value that is equal to a predetermined target VCD; a value that is −2.5% the target VCD; a value that is −5% the target VCD; a value that is −10% the target VCD; and the like. In such an example, if the ACU 155 determines that a measured VCD value is equal to greater than a predefined VCD-based trigger value, then the ACU 155 treats that condition as a trigger event for actuating the pump 130 to effect a fluid flow through the fluid line 135, such that a culture medium in the bioreactor 140 is automatically inoculated with a cell culture volume from the expansion chamber 110.

The ACU 155 may store any number of predefined trigger values, establishing conditions for any number of trigger events. For example, a first trigger value may be set based on a target VCD and a second trigger value may be set based on a minimum lactate value. The VCD-based trigger value may represent a target VCD for use in inoculating the bioreactor 140, such as described earlier, while the lactate-based trigger value may identify a minimum lactate level that has been predetermined to signal a change in cell growth state. Such a lactate-based trigger value may be set to a value that is equal to a predetermined minimum lactate level; a value that is +2.5% the minimum lactate level; a value that is +5% the minimum lactate level; a value that is +10% the minimum lactate level; and the like. In such an example, the ACU 155 may be adapted to auto-inoculate the bioreactor 140 upon detecting either trigger event, such that the system 10 inoculates the bioreactor 140 once the predefined VCD trigger value is reached, though may trigger auto-inoculation at a lower VCD if there is detected a lactate level measurement that is equal to or less than the predefined lactate-based trigger value, thereby ensuring inoculating of the bioreactor 140 prior to a change in cell growth state.

As a further example, the ACU 155 may operate with a first trigger value based on a target VCD, a second trigger value based on any processing variable that has been predetermined in advance as indicative of a change in cell growth state, and a third trigger value based on a model predicted VCD. A model predicted VCD-based trigger value may be set to a value equal to a maximum model predicted VCD that is deemed acceptable for a given seed train process; a value that is −2.5% the maximum cell growth rate; a value that is −5% the maximum cell growth rate; a value that is −10% the maximum cell growth rate; and the like. In such an example, the ACU 155 may be adapted to auto-inoculate the bioreactor 140 upon detecting any of the trigger events, such that the system 10 inoculates the bioreactor 140 once the predefined VCD trigger value is reached, though may trigger inoculation at a lower VCD if there is detected a processing variable value that satisfies a condition that has been predetermined as indicative of a change in cell growth state, with the added precaution that an earlier auto-inoculation is also triggered if there is detected a model predicted VCD that is equal to or greater than a predefined model predicted VCD trigger value. In this way, if a cell culture begins to experience increasing model predicted VCD prior to detection of a predefined VCD trigger value, and without detection of any other processing variable that provides a warning of a change in cell growth state, then the system may trigger an auto-inoculation before an unacceptable cell growth state is incurred.

The ACU 155 may operate with any number of predefined trigger values, based on any number of different process variables, which may include, without limitation, any one or combination of: one or more nutrients (such as amino acids and vitamins); lactate; co-factors; growth factors; cell growth rate; pH; oxygen; nitrogen; viable cell count; cell death count; acids; bases; cytokines; antibodies; and metabolites.

The computer system 150 may also have controls that enable changes to the system, including the ACU 155, in real time from a platform interface. For instance, there may be an interface that allows a user to select one or more trigger conditions based on a number of different processing variables (e.g., a VCD-based trigger condition; a lactate-based trigger condition; a cell growth rate-based trigger condition; etc.); to input a desired value for use as a predefined set point value in a trigger condition (e.g., trigger values based on a target VCD; a minimum lactate level; a maximum cell growth rate, etc.); and to adjust one or more pre-set trigger values. The ACU 155 should be capable of responding to a change in one or more predefined trigger values to adjust the conditions under which auto-inoculation is triggered.

In a first seed train process, Raman spectral data was used to generate a multivariate model based on process variable measurements throughout the ranges of 450-1800 cm⁻¹ and 2600-3100 cm⁻¹, while excluding measurements in the range of 1800<x<2600 cm⁻¹. In parallel with the Raman spectral measurements, offline spectral measurements were also taken using a BioProfile Flex® Analyzer. Raman spectral measurements were taken once every 15-60 minutes, whereas the offline process variables measurements were taken once at each of approximately 4 hours, 24 hours, 48 hours, and 72 hours following introduction of the cryopreserved cell stock into the expansion chamber 110. FIG. 5 shows data from the two spectral measurements, together with a target VCD (4.0×10⁶ cells/mL) for inoculation of the bioreactor 140.

As seen in FIG. 5, the two spectral data sets were relatively in agreement with one another at the 24 hour mark, corresponding with the second offline measurement, though began to diverge at around the 32 hour mark. Though the two data sets again converge at around the 72 hour mark, corresponding with the fourth offline measurement, there was observed a maximum offset of approximately 2.0×10⁶ cells/mL at about the 48 hour mark, corresponding with the third offline measurement. This offset is significant. For example, when targeting an inoculation VCD of 4.0×10⁶ cells/mL, if the ACU 155 were to rely on the Raman spectral data, then auto-inoculation of the bioreactor 140 would occur at approximately 44 hours. However, based on the offline spectral data, auto-inoculation at 44 hours would be premature as the VCD at that time would in fact have been approximately 2.5×10⁶ cells/mL. Instead, presuming the offline spectral data is accurate, the target VCD for inoculation would not be achieved until approximately 60 hours. As such, auto-inoculation based on the Raman spectral data would have occurred at a sub-optimal VCD, which could lead to a substantial shortfall in the output of the production process.

An offset such as that in FIG. 5 can be problematic for accurate and reliable inoculation of a bioreactor. For example, if there is an error in the Raman spectral data, then the auto-inoculation may be executed too early, before an optimal VCD is in fact reached. On the other hand, when the offline process variable measurements are taken only once every several hours, manual inoculation could be executed too late, such as when manual inoculation is not performed at the 48 hour mark when the VCD is below target (as in FIG. 5), and is instead performed at the 72 hour mark after the target VCD had been exceeded (FIG. 5).

In seeking to improve accuracy and reliability of the Raman spectral measurements, a cell spiking study was undertaken with a CHO cell suspension at six different densities, as identified in the following table I:

TABLE I

| Target Cell | Actual Cell Density |
|---|---|
| Density (×10⁶) | (NovaFLEX ®, ×10⁶) |
| 1 | 1.03 |
| 4 | 3.85 |
| 7 | 7.68 |
| 10 | 9.39 |
| 13 | 13.09 |
| 16 | 15.65 |

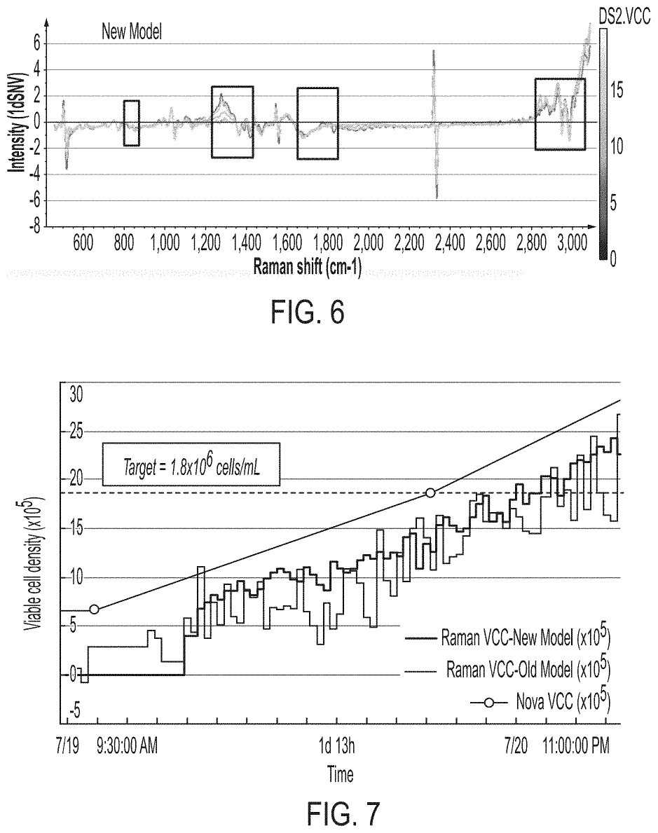

Raman spectral measurements were taken of the six different cell densities in triplicate, replicating the scan time used in upstream Raman data collection, and a variable influence on prediction (VIP) plot was used to identify those wavelength regions of the Raman spectral data that were observed to correlate the strongest with the cell density measurements. From this study the wavelength regions of 800-850 cm⁻¹; 1260-1470 cm⁻¹; 1650-1840 cm⁻¹; and 2825-3080 cm⁻¹ were identified as most accurately informing cell density (FIG. 6).

FIG. 7 shows the results of a comparative example between measurements obtained from the two spectral data sets, with the first being based on Raman spectral measurements taken in accord with conventional practices, and the second being based on Raman spectral measurements taken in accord with the innovative practices, with both data sets plotted relative to offline spectral data. The conventional Raman spectral measurements were taken across wavelength ranges of 450-1800 cm⁻¹ and 2600-3100 cm⁻¹, whereas the innovative Raman spectral measurements were taken across the wavelength ranges of 800-850 cm⁻¹; 1260-1470 cm⁻¹; 1650-1840 cm⁻¹; and 2825-3080 cm⁻¹. As can be seen, though both Raman data sets display some offset from the offline measurements, the measurements taken in accord with the innovative practices herein displayed noticeably less sample-to-sample variability.

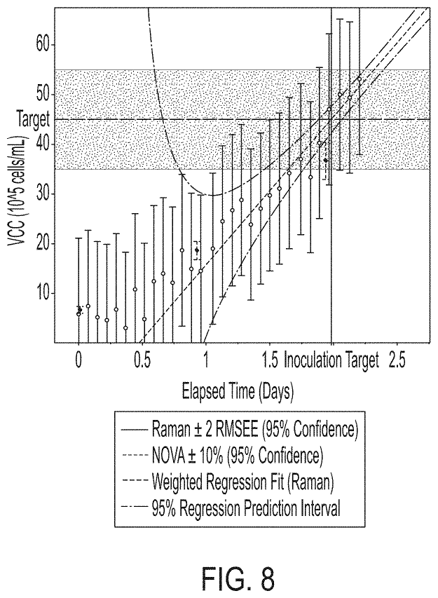

In a further aspect of the present invention, in addition to the computer system 150 retaining a predicted process variable from the multivariate model, the computer system 150 may retain a time series of predicted process variables, either in expansion chamber 110 or the bioreactor 140. This time series may be subject to a noise reduction technique, which may be predictive or retrospective. When incorporating a noise reduction technique, the ACU 155 may be a locally weighted regression model, as in Cleveland, W.S., Robust Locally Weighted Regression and Smoothing Scatterplots. Journal of the American Statistical Association, Vol. 74, No. 368 (1979): 829-836 which is incorporated by reference in its entirety. For example, the weight function can be a fifth-order polynomial that deemphasizes early points in the batch and emphasizes recent points. Prior knowledge informs the intuition that cell growth in the N−1 is sigmoidal, which implies that the middle growth region is approximately linear. Local regression models can estimate this linearity and extrapolate to update a predicted inoculation time, and also calculate a time between measurements where the estimated process value will be equal to the trigger value. This approach reduces the variation in the prediction of a single Raman measurement as shown in FIG. 8.

Although the present invention is described with reference to particular embodiments, it will be understood to those skilled in the art that the foregoing disclosure addresses exemplary embodiments only; that the scope of the invention is not limited to the disclosed embodiments; and that the scope of the invention may encompass additional embodiments embracing various changes and modifications relative to the examples disclosed herein without departing from the scope of the invention as defined in the appended claims and equivalents thereto.

To the extent necessary to understand or complete the disclosure of the present invention, all publications, patents, and patent applications mentioned herein are expressly incorporated by reference herein to the same extent as though each were individually so incorporated. No license, express or implied, is granted to any patent incorporated herein.

The present invention is not limited to the exemplary embodiments illustrated herein, but is instead characterized by the appended claims.

What is claimed is:

1. A system for controlling a seed train process, comprising:
an expansion chamber for receiving an initial cell stock for expansion into a viable cell culture;
a bioreactor in fluid communication with the expansion chamber for receiving a viable cell culture;
a pump for effecting transfer of a viable cell culture from the expansion chamber to the bioreactor through a fluid communication path between the expansion chamber and the bioreactor;
a Raman spectrometer having at least one probe for monitoring the cell expansion process within the expansion chamber using Raman spectrometry, the Raman spectrometer being adapted to generate Raman spectral data;
a multivariate model that provides predictions of process variables based on Raman spectral data; and
a computer system in signal communication with the Raman spectrometer for receiving Raman spectral data, and in signal communication with the pump for controlling operation of the pump for effecting transfer of a viable cell culture from the expansion chamber to the bioreactor,
wherein the Raman spectrometer is adapted to generate Raman spectral data and a multivariate model that provides predictions of one or more process variables, and the computer system is adapted to compare the process variable measurements to one or more predefined process set points to determine if one or more process variable measurements have satisfied a predefined trigger value, and
wherein the computer system is adapted, upon determining that a process variable measurement in the Raman spectral data has satisfied a predefined trigger value, to control the pump to execute an auto-transfer of a cell culture volume from the expansion chamber to the bioreactor.

2. The system according to claim 1, wherein
the computer system processes Raman spectral data received from the Raman spectrometer to generate a multivariate model of the one or more process variables.

3. The system according to claim 2, wherein
the computer system generates a partial least squares regression model.

4. The system according to claim 3, wherein
the computer system is adapted, when comparing process variable predictions from the multivariate model to one or more predefined process set points, to use process variable measurements from a plurality of predefined isolated regions of the Raman spectral data.

5. The system according to claim 4, wherein
the computer system uses process variable measurements from Raman spectral data in the wavelength regions of 800-850 cm⁻¹; 1260-1470 cm⁻¹; 1650-1840 cm⁻¹; and 2825-3080 cm⁻¹.

6. A method of auto-inoculating a bioreactor using a system according to claim 1, comprising:
expanding a cell stock in the expansion chamber;
generating Raman spectral data, using the Raman spectrometer, to provide data to a multivariate model that predicts one or more process variables of the cell expansion in the expansion chamber;
the computer system comparing process variable predictions from the multivariate model with predefined process set points at the computer system;
the computer system controlling the pump to auto-inoculate the bioreactor with a viable cell culture from the expansion chamber when the computer system determines that one or more process variable predictions from the multivariate model satisfies a predefined trigger value.

7. The method according to claim 6, wherein
the predefined trigger value is a viable cell density value.

8. The method according to claim 7, wherein
the predefined trigger value is set to a viable cell density value that is equal to or within a range of −10% of a predetermined target viable cell density.

9. The method according to claim 6, wherein
the predefined trigger value is a lactate level value.

10. The method according to claim 9, wherein
the predefined trigger value is set to a lactate level value that is equal to or within a range of +10% of a predetermined minimum lactate level.

11. The method according to claim 6, wherein
the predefined trigger value is a model predicted VCD.

12. The method according to claim 11, wherein
the predefined trigger value is set to a model predicted VCD value that is equal to or within a range of −10% of a predetermined maximum cell growth rate.

13. The method according to claim 6, wherein
the computer system stores a first predefined trigger value based on a predetermined viable cell density, and stores a second predefined trigger value based on a predetermined processing variable other than viable cell density, and
the computer system is adapted to control the pump to auto-inoculate the bioreactor with a viable cell culture from the expansion chamber when the computer system determines that a process variable prediction from the multivariate model satisfies either the first or second predetermined trigger value.

14. The method according to claim 6, wherein
the second predetermined trigger value is a lactate level value.

15. The method according to claim 6, wherein
the predefined trigger value is a model predicted VCD value.

16. The method according to claim 6, wherein
the computer system processes Raman spectral data received from the Raman spectrometer to generate a multivariate model of the one or more process variables, and obtains the process variable measurements from the multivariate model for comparison with the predefined trigger values.

17. The method according to claim 16, wherein
the computer system generates a partial least squares regression model.
